(19) United States
(12) Patent Application Publication
(10) Pub. No.: US 2004/0106187 A1
(43) Pub. Date: Jun. 3, 2004

(54) Composition for the culturing of Phellinus linteus mycelium

(76) Inventors: Nam-Doo Hong, Seoul (KR); Soo Muk Cho, Gyeonggi-do (KR); Jae Kuk Yoo, Daejeon (KR)

Correspondence Address: BACHMAN & LAPOINTE, P.C., 900 CHAPEL STREET, SUITE 1201, NEW HAVEN, CT, 06510, US

(21) Appl. No.: 10/421,288

(22) Filed: Apr. 23, 2003

(30) Foreign Application Priority Data
Nov. 30, 2002 (KR) 2002-75631

(51) Int. Cl. C12N 1/18; C12N 1/16

(52) U.S. Cl. 435/254.3; 435/256.8

(57) ABSTRACT

The present invention relates to a medium composition for the culture of Phellinus linteus strain producing galactomannoglucan and a method for the culture thereof using the same, more particularly, relates to a medium composition for the culture of Phellinus linteus mycelium composed of CSP (corn steep powder), yeast extract, soy peptone, malt extract and distilled water and a method for the culture of Phellinus linteus strain, in which a medium is prepared by autoclaving with steam pressure for 50-60 minutes and cooling, tissues of fruit body or spores of the strain are sub-cultured on agar medium, and then inoculates the sub-cultured products to the medium prepared with the above composition. By using the medium composition of the present invention, the culture and extraction of Phellinus linteus mycelium producing galactomannoglucan having an excellent medicinal effect becomes easier and high productivity resulted from upgraded fermenting efficacy and shortening of the culture time and the mass-production of Phellinus linteus mycelium with less expense are expected.

### FIELD OF THE INVENTION

The present invention relates to a composition for culturing of a specific strain and a method for culturing of the specific strain using the same, more particularly, to a composition for culturing of Phellinus linteus strain producing galactomannoglucan and a method for culturing of Phellinus linteus strain using the same.

### BACKGROUND ART OF THE INVENTION

Although various methods such as chemotherapy, radiotherapy and surgical operation have been tried so far to treat cancer, the treatment is limited in use because of side effects and limited curing effect. Thus, based on the thought that the conventional methods having direct cytotoxicity that was harming human should be substituted or complemented, attempts have been made to treat cancer effectively and without damaging human body by promoting immune response to cancer cells.

Anti-cancer polysaccharides produced by Basidiomycotina have been reported to be very safe with less side effects and to stimulate the function of human immune system, showing the excellent anti-cancer effect. Especially, many kinds of mushrooms belonging to order Aphyllophorales, as a kind of Basidiomycotina, have been known to be available for the treatment of many types of diseases including cancer, so that they have been used as herb medicines or folk remedies. Among them, Phellinus linteus according to family Polyporaceae genus Phellinus, called as “Sanghwang” (Linteus) in Chinese medicine, is very rare perennial hard woody mushroom that grows on the stump of an old mulberry tree. When observed the section of Phellinus linteus, it had a characteristic dark yellow color and an annual ring-like pattern on its upper fruiting body. As a very valuable herb medicine, Phellinus linteus has been used as an anhidrotics or for women's diseases from ancient times and was once confirmed to have a strong anti-cancer activity in 1960s.

As for the existing inventions that related to polysaccharides produced by Phellinus linteus strain, Korea Patent #92-2658 and #95-29192 were reported. In addition, the present inventors had separated a novel polysaccharide increasing immune activity from Phellinus linteus strain (KCTC 0399BP) that was totally different from the other established strains in anti-cancer effect and in microbiological characteristics, and proved its component was galactomannoglucan in earlier studies (Korea Patent #98-15617).

Even though the fact that many components including the above polysaccharides of Phellinus linteus have a great effect on cancer treatment was well recognized, Phellinus linteus cannot be in much use since it is difficult not only to obtain fruit bodies of Phellinus linteus but also to separate and culture its mycelium.

There are two methods to manufacture food or medicine with the extracted effective medical components from such rare wild mushroom as Phellinus linteus. One is to use fruit bodies as a raw material after collecting fruit bodies or culturing fruit bodies and the other is to culture mycelia after separating mycelia from fruit bodies, followed by extracting effective components from the cultured mycelia.

However, the way to use collected fruit bodies brings the exhaustion of natural resources and the destruction of ecosystem. Besides, it is absolutely impossible to obtain required amount of such rare Basidiomycetes as Phellinus linteus. The way to obtain fruit bodies by culturing is not preferable either since it requires special facilities and high cost and the production period is limited even though culturing period takes long, three to six months.

Meanwhile, the way to culture mycelia after separating from fruit bodies seems to solve the above problems, but the method cannot be in general use because of technical difficulties, that is, it is difficult to purely separate mycelia, it requires particular culture conditions, it has high probability of contamination with other bacteria owing to the low growth speed and it is difficult to facilitate proper fermentor and culture conditions for the culture without contamination.

There is a relative report concerning the method for culturing mycelia of mushrooms that Phellinus linteus strain was mass-produced by being cultured in conventional medium containing yeast extract, peptone and KH₂PO₄ (Korea Patent #95-29192). However, the culture at that time was done in liquid medium. When mycelia are cultured in such liquid medium, it can hardly produce various secondary metabolites of mushrooms except polysaccharides. Thus, it is impossible to expect to obtain effective medical components from the secondary metabolites. In the mean time, when a mushroom is cultured on solid medium, fruit bodies and mycelia containing effective medical components can be obtained as the secondary metabolites of the mushroom.

Phellinus linteus strain of the present invention cannot be cultured on conventional solid media such as peptone medium, Mayer's medium, yeast extract medium and a medium containing sawdust and glucose (Korea Patent #88-2475, #83-1909), so that anyone has not been able to succeed in artificial culture for the mass-production of Phellinus linteus strain so far.

In order to solve the above problems, the present inventors have studied and then established a novel method for artificial culture of Phellinus linteus strain (KCTC 0399BP) with liquid medium that is different from the conventional media in its composition, in which Phellinus linteus strain was different from the other strains in microbiological characteristics and had an excellent anti-cancer activity by producing galactomannoglucan, comparing to other collected domestic Phellinus linteus. And the present inventors completed the present invention by confirming that the liquid medium of the present invention was good for the culture and extraction of Phellinus linteus mycelium producing galactomannoglucan having an excellent medicinal effect, and contributed to shortening the culture time and increasing productivity of fermentation.

### DETAILED DESCRIPTION OF THE INVENTION

It is an object of the invention to provide liquid medium compositions for the culture of Phellinus linteus strain producing galactomannoglucan and a method for the culture of Phellinus linteus strain using the same.

To achieve the above object, the present invention provides liquid medium compositions for mass-production of Phellinus linteus mycelium in which the liquid medium is composed of corn steep powder (CSP), soy peptone, malt extract and distilled water.

The present invention also provides a method for the culture of Phellinus linteus mycelium using the liquid medium composed of the above components.

Further features of the present invention will appear hereinafter.

The present invention provides liquid medium compositions for the mass-production of Phellinus linteus mycelium.

Phellinus linteus used in this invention is a strain deposited at Gene Bank of Korea Research Institute of Bioscience and Biotechnology, an international depositary authority, with the accession number KCTC 0399BP and has microbiological characteristics as follows (Table 1).

TABLE 1
Microbiological characteristics of Phellinus linteus
(KCTC 0399BP) strain

| Index | Characteristic |
|---|---|
| Shape of | Sessile, Dimidiate to elongate, Woody |
| fruit body | Semicircular flat mustard having a width |
| | of 10 cm, Dark brown surface, Yellowish |
| | brown or dark brown back |
| | Multilayered pore |
| Spore | Subglobose, Pale golden brown |
| | Size: 4.5˜6 × 4˜5 μm |
| Seta body | Abundant, Thick walled |
| | Size: 20˜40 × 8˜12 μm |

The constitution rate of a composition for the culture of Phellinus linteus strain of the present invention is characteristically different from that of conventional medium, even though such nutrients that ordinary microorganisms are using for the growth are also used. Particularly, the composition of the present invention is preferably prepared by adding 30˜100 g of CSP, 0.003˜5 g of yeast extract, 0.0003˜0.5 g of soy peptone and 0.003˜5 g of malt extract to 1 l of distilled water. It is more preferable to add 50˜80 g of CSP, 0.05˜0.8 g of yeast extract, 0.005˜0.08 g of soy peptone and 0.05˜0.8 g of malt extract to 1 l of distilled water for the preparation of the composition. CSP, a major element of the medium composition of the present invention, is extracted from corns, and its ingredients can be used not only as an effective carbon source but also as a useful nitrogen source owing to its rich protein content. In addition, CSP contains plenty of various sugars, organic acids, vitamins and inorganic elements. Other trace elements such as essential amino acids, minerals, vitamins, etc that CSP cannot supply are provided by yeast extract, soy peptone or malt extract for the culture of the above strain. Yeast extract contains plenty of amino acids and vitamins, soy peptone includes lots of amino acids and peptides, and malt extract is rich in carbohydrates. By using CSP as a major ingredient for the medium composition of the present invention, more active Phellinus linteus mycelium can be obtained that produces galactomannoglucan having more than 20% higher anticancer and immune activity, comparing to when other conventional medium is used.

The above liquid medium is a kind of natural medium whose composition rate of nutrients is more complicated than a, synthetic medium having exact and clear chemical composition rate, though, industrial supply of the liquid medium is easy regardless of season or time and the cost for the preparation thereof is not expensive. Moreover, anybody can prepare the liquid medium without difficulties or special attention on pH regulation and the generation of precipitation. The liquid medium of the present invention can produce more mycelium and galactomannoglucan than any other medium including a synthetic medium and even excluded the weakness of a natural medium such as the unstable result of culture and productivity. Further, scale-up of culture is possible producing the same good results as the culture of laboratory scale. Therefore, the mass-culture of Phellinus linteus mycelium and high productivity of galactomannoglucan can be obtained by using the liquid medium of the present invention.

The present invention also provides a method for the culture of Phellinus linteus mycelium using the liquid medium composed by the above compositions.

Particularly, the present invention provides a method for the culture of Phellinus linteus strain characterized by that the medium was autoclaved by steam pressure for 50-60 minutes and then cooled, and tissues of fruit body or spores of Phellinus linteus strain were sub-cultured on an agar medium, after which the culture products were cultured in the liquid medium prepared above.

There is no appointed temperature for the culture but it is preferable to culture at 15-35° C. Culture time depends on medium composition or culture temperature but generally 20-200 days would be preferable and 50-120 days of culture would be more preferable.

Agar medium is used for the sub-culture of Phellinus linteus strain to obtain primary culture products. For example, a medium was selected from a group consisting of PSA (potato sucrose agar) medium, PDA (potato dextrose agar) medium, MEA (malt extract agar) medium, QEA (quercus extract agar) medium, PEA (poplar extract agar) medium, RBEA (rice bran extract agar) medium, WBEA (wheat bran extract agar) medium, MCM (mushroom complete medium) medium, CDA (Czapedox agar) medium, SCA (Spawn complex agar) medium, OMA (oatmeal agar) medium. Especially, MCM medium is beneficial to an outstanding growth of the strain.

The liquid culture process of Phellinus linteus strain is explained below.

First, for the liquid culture of Phellinus linteus strain, The present inventors transfer tissues of fruit body or spores of the strain to an agar medium composed of potato, malt extract, quercus sawdust, poplar sawdust, rice bran and oatmeal, and then culture the same at 15˜35° C. for weeks. The above process is repeated 2-3 times. After confirming that there is no contamination, The present inventors transfer the same into the liquid medium and continue to culture at 13˜36° C. for 20-200 days.

At this time, most of the major ingredients of CSP are absorbed into Phellinus linteus mycelium, that is, the ingredients are converted to Phellinus linteus mycelium. While passing through the maturity, various and unique components of Phellinus linteus are generated, resulting in the generation of mycelium having unique dark yellow color of Phellinus linteus. Therefore, the extracted available components by using those mycelium are different from that obtained by other general liquid culture. Moreover, the mass-production of available medicinal components of galactomannoglucan from Phellinus linteus mycelium is possible with the liquid medium of the present invention, in which productivity is like or more than that of a general solid medium. Particularly, the present inventors add required amount of water to the mycelium cultured in the liquid medium, followed by heat extraction. The same is filtered with a centrifuge. High molecular substances containing galactomannoglucan is extracted after going through the process of ethanol precipitation and is dialyzed with the obtained solution. The present inventors inject the extracted substances into test animals for the cell culture and then investigate the anticancer activity.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Preparation of Agar Media

Various agar media composed of potato, malt extract, quercus sawdust, poplar sawdust, rice bran, wheat bran or oatmeal were prepared (Table 2).

TABLE 2
Composition of an agar medium

| Medium | Composition (g/l) |
|---|---|
| PSA medium | Potato 200, Sucrose 20, Agar 20 |
| (Potato sucrose agar) | |
| PDA medium | Potato 200, Dextrose 20, Agar 20 |
| (Potato dextrose agar) | |
| MEA medium | Malt extract 20, Dextrose 20, |
| (Malt extract agar) | Peptone 1, MgSO₄.7H₂O 0.5, |
| | Agar 20 |
| QEA medium | Quercus sawdust 100, |
| (Quercus extract agar) | Dextrose 20, Agar 20 |
| PEA | Poplar sawdust 100, Dextrose 20, |
| (Poplar extract agar) | Agar 20 |
| RBEA | Rice bran 30, Agar 20 |
| (Rice bran extract agar) | |
| WBEA | Wheat bran 30, Agar 20 |
| (Wheat bran extract agar) | |
| MCM | Dextrose 20, Peptone 2, Yeast |
| (Mushroom complete | extract 2, MgSO₄.7H₂O 0.5, KH₂PO₄ |
| medium) | 0.46, Agar 20 |
| CDA | Dextrose 20, MgSO₄.7H₂O 0.5, |
| (Czapedox agar) | KH₂PO₄ 1, Agar 20 |
| SCA | Quercus 60, Poplar sawdust 60, |
| (Spawn complex agar) | Rice bran 20, Agar 20 |
| OMA | Oatmeal 30, Agar 20 |
| (Oatmeal agar) | |

### Example 2-9

### Preparation of Liquid Media

Liquid media composed of CSP, yeast extract, soy peptone, malt extract and distilled water were prepared. Particularly, the present inventors prepared liquid media by adding CSP, yeast extract, soy peptone and malt extract to 1 l of distilled water in the ratio represented in Table 3.

TABLE 3

Medium composition (/Distilled water 1 l)

| | | Yeast | Soy | Malt |
|---|---|---|---|---|
| | CSP | extract | peptone | extract |
| | (g) | (g) | (g) | (g) |
| Example 2 | 80 | 0.1 | 0.01 | 0.1 |
| Example 3 | 80 | 0.003 | 0.0003 | 0.003 |
| Example 4 | 80 | 5 | 0.5 | 5 |
| Example 5 | 80 | 0.8 | 0.08 | 0.8 |
| Example 6 | 80 | 0.05 | 0.005 | 0.05 |
| Example 7 | 80 | 0.1 | .01 | 0 |
| Example 8 | 80 | 0.1 | 0 | 0.1 |
| Example 9 | 80 | 0 | 0.01 | 0.1 |

### Example 10

### Culture of Phellinus Linteus Strain

Transplanted tissues of fruit body or spores of Phellinus linteus strain (KCTC 0399BP) onto the agar medium prepared in the above Example 1 and cultured the same at 20±2° C. for 30 days. Repeated the culture process three times. After confirming that there was no contamination, transferred the mycelium into the liquid medium prepared in the above Examples 2-9 and continued to culture at 25±2C for 100 days. On completing the culture, filtered the mycelium, washed the same with distilled water and then measured the density of hypha (volume of hypha per liquid medium) and dry weight—of mycelium. The results were represented in Table 4 (the results were mean values of 4 round experiments).

TABLE 4

| | Density of hypha | Dry weight of mycelium |
|---|---|---|
| | (v/v) | (after 100 day culture) |
| Example 2 | 0.07 | 20 ± 0.5 g |
| Example 3 | 0.056 | 16 ± 1.0 g |
| Example 4 | 0.063 | 18.0 ± 1.0 g |
| Example 5 | 0.068 | 19.5 ± 0.8 g |
| Example 6 | 0.066 | 19.0 ± 0.7 g |
| Example 7 | 0.025 | 6.5 ± 0.7 g |
| Example 8 | 0.034 | 10 ± 1.0 g |
| Example 9 | 0.022 | 6.0 ± 0.5 g |

As shown in Table 4, when the medium was composed of CSP (major component), yeast extract, soy peptone and malt extract, the mycelium grew faster and the density of hypha was higher. But in the case of losing one of supplementary nutrients, the growth of Phellinus linteus strain (KCTC 0399BP) was dramatically decreased.

Following experiments were performed to confirm galactomannoglucan content and the medicinal effect thereof obtained from Phellinus linteus strain (KCTC 0399BP) cultured in the medium composition of the present invention.

### Experimental Example 1

### Extraction of Galactomannoglucan

The present inventors extracted the mycelium of Phellinus linteus KCTC 0399BP obtained in the above Example 10 as following procedures. Particularly, put 100 g of mycelium into 500 ml of distilled water and heated at 90˜100° C. for 2 hours for the extraction, after which removed mycelium cake and recovered only hot water extracts. After vacuum-concentrating the hot water extracts until the volume came to 100 ml, added 4 volumes of 95% ethanol and then left the same for overnight, followed by centrifuging at 3,000 rpm for 30 minutes. Dissolved the obtained extracts again in 100 μl of water and dialyzed for 3 days using a dialyzing membrane that is able to dialyze materials under 3,000 molecular weight. Finally, obtained about 3 g of high molecular extracts containing galactomannoglucan by freeze drying at −70° C.

### Experimental Example 2

### Anticancer Effect of the Extracted Galactomannoglucan

The present inventors investigated the anticancer activity of galactomannoglucan extracted in the above Experimental Example 1 in vivo and in vitro. Particularly, used specific pathogen free (SPF) BDF1 mice (Korea Research Institute of Bioscience and Biotechnology; male, 23-28 g) as test animals. Diluted sarcoma-180 cells with PBS and adjusted the density to 5×10⁶ cells/ml. Injected 0.2 ml of the cell suspension (1×10⁶ cells) into the peritoneal cavity of the mice and administered galactomannoglucan (10 mg/kg or 30 mg/kg) for 3 weeks. Then, observed the mice for 35 days.

As a result, mice of a group treated with galctomannoglucan simultaneously with transplantation of cancer cells survived 25 days while mice of a control group not treated with galctomannoglucan survived 19 days at average (Table 5). Thus, it was confirmed that the galactomannoglucan had anticancer activity.

TABLE 5

| | Dose | Mice | Mean survival | ILS |
|---|---|---|---|---|
| Group | (mg/kg) | number | time (day) | (%) |
| Control | — | 10 | 19.1 ± 0.81 | — |
| Galatomannoglucan | 10 | 10 | 24.7 ± 1.48 | 39.2 |
| | 30 | 10 | 24.8 ± 2.34 | 29.4 |

### INDUSTRIAL APPLICABILITY

As described hereinbefore, by using the liquid medium composition and the culture method of the present invention, the culture and extraction of Phellinus linteus mycelium producing galactomannoglucan having an excellent medicinal effect become easier than when using the conventional liquid medium or solid medium, and also high productivity resulted from upgraded fermenting efficacy and shortening of culture time and mass-production of Phellinus linteus mycelium with less expense are expected.

What is claimed is:

1. A medium composition for the culture of Phellinus linteus mycelium composed of CSP, yeast extract, soy peptone, malt extract and distilled water.

2. The composition as set forth in claim 1, wherein the Phellinus linteus is Phellinus linteus KCTC 0399BP.

3. The composition as set forth in claim 1, wherein the composition is prepared by adding 30-100 g of CSP, 0.003-5 g of yeast extract, 0.0003-0.5 g of soy peptone and 0.003-5 g of malt extract into 1 l of distilled water.

4. The composition as set forth in claim 3, wherein the composition is prepared by adding 50-80 g of CSP, 0.05-0.8 g of yeast extract, 0.005-0.08 g of soy peptone and 0.05-0.8 g of malt extract into 1 l of distilled water.

5. A method for the culture of Phellinus linteus strain, wherein a medium is prepared by autoclaving with steam pressure for 50-60 minutes and cooling, tissues of fruit body or spores of the strain are sub-cultured on an agar medium, and then the sub-cultured products are inoculated into the liquid medium prepared with the composition of claim 3 or claim 4.

6. The method for the culture of Phellinus linteus strain as set forth in claim 5, wherein the culture temperature of an agar medium and a liquid medium is 15-35° C.

7. The method for the culture of Phellinus linteus strain as set forth in claim 6, wherein the culture time is 50-120 days.
